(19) United States
(12) Patent Application Publication
(10) Pub. No.: US 2026/0066106 A1
(43) Pub. Date: Mar. 5, 2026

(54) METHOD FOR CONFIGURING A DEVICE WITHIN A MEDICAL NETWORK

(71) Applicant: Siemens Healthineers AG, Forchheim (DE)

(72) Inventors: Björn Nolte, Fürth (DE); Yoshihisa Shinagawa, Downingtown, PA (US); Halid Yerebakan, Carmel, IN (US)

(21) Appl. No.: 19/280,522

(22) Filed: Jul. 25, 2025

(30) Foreign Application Priority Data
Aug. 29, 2024 (DE) 10 2024 124 715.0

(51) Int. Cl. G16H 40/63; G06N 3/09

(52) U.S. Cl. CPC G16H 40/63; G06N 3/09

(57) ABSTRACT

A framework for configuring a device within a medical network. The medical network comprises at least one other device for communicating with the device via a network connection. The framework includes collecting configuration data regarding the device, the network connection and/or the at least one other device. A neural network is trained, in a self-supervised manner, using the collected configuration data. The neural network may be pretrained using configuration data from a plurality of medical networks. The device is configured depending on the trained neural network.

### CROSS-REFERENCE TO RELATED APPLICATION

This application claims the benefit of priority from German DE Application No. 10 2024 124 715.0, filed on Aug. 29, 2024, the contents of which are incorporated by reference.

### TECHNICAL FIELD

The present framework relates to a method for configuring a device within a medical network, to a computer program product, to a system for configuring a device within a medical network, and to a medical device configured to be operable in a medical network.

### BACKGROUND

Hospitals comprise large information technology (IT) networks. Oftentimes, new devices need to be added to such a network, or an existing device needs to be updated. Due to the complexity of such networks, integrating a new device or upgrading an existing device may require a vast amount of configuration to be done. For example, the hospital's IT administrator must read the device's documentation and must have, in addition, a good understanding of other systems or entities in the network.

In many cases, the new device or the device to be upgraded comes with a user interface tailored to that device. Understanding the user interface and entering the correct configuration data can be a challenging task. Also, knowledge of the other systems and entities in the network and their interplay with the new device or device to be upgraded may not be fully known. This makes the integration of a new device or upgrading an existing device an error-prone task. Even more, troubleshooting an incorrectly configured new device or upgraded device may be time-consuming and difficult. This is even more true where it is unknown whether it is the newly added or upgraded device or a system or entity in communication with said device which is causing the fault.

### SUMMARY

Disclosed herein is a framework for configuring a device within a medical network. The medical network comprises at least one other device for communicating with the device via a network connection. The framework includes collecting configuration data regarding the device, the network connection and/or the at least one other device. A neural network is trained, in a self-supervised manner, using the collected configuration data. The neural network may be pretrained using configuration data from a plurality of medical networks. The device is configured depending on the trained neural network.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1 shows a block diagram of a client server architecture embodying a medical network;

FIG. 2 shows a block diagram of a system for configuring a device within the medical network of FIG. 1;

FIG. 3 illustrates a flowchart of an embodiment of a computer-implemented method for configuring a device within the medical network of FIG. 1;

FIG. 4 shows an example of an excerpt of a ports map documentation;

FIG. 5 shows example configurations for connecting DICOM and FHIR/HL7 systems;

FIG. 6 shows a medical network connected to a vendor network according to an embodiment;

FIG. 7 shows an example of a firewall rules configuration;

FIG. 8 shows a view of a DICOM discovery service protocol;

FIG. 9 shows an example configuration of preprocessing rules;

FIG. 10 shows an example of a configuration user interface for handling of prior clinical studies;

FIG. 11 shows an example of a configuration file; and

FIG. 12 shows ways of configuring a device according to various embodiments.

In the Figures, like reference numerals designate like or functionally equivalent elements, unless otherwise indicated.

### DETAILED DESCRIPTION

Hereinafter, embodiments for carrying out the present invention are described in detail. The various embodiments are described with reference to the drawings, wherein like reference numerals are used to refer to like elements throughout. In the following description, for purpose of explanation, numerous specific details are set forth in order to provide a thorough understanding of one or more embodiments. It may be evident that such embodiments may be practiced without these specific details.

Independent of the grammatical term usage, individuals with male, female or other gender identities are included within the term.

It is one object of the present framework to facilitate the configuration of a device within a medical network. This object is solved with the subject matter of the independent claims. Alternative and/or preferred embodiments are the subject of the dependent claims.

According to a first aspect, there is provided a method for configuring a device within a medical network, the medical network comprising at least one other device for communicating with the device via a network connection, the method comprising:
a) collecting configuration data regarding the device, the network connection and/or the at least one other device;
b) training, in a self-supervised manner, a neural network using the collected configuration data, the neural network being pretrained prior to step a) using configuration data from a plurality of medical networks; and
c) configuring the device depending on the trained neural network.

One idea of the present framework is to use a neural network to bridge the knowledge gap for the clinical IT administrator by assisting during the configuration of a device newly added to a medical network or updating or upgrading an existing device within that network.

A neural network is, preferably, trained to assist with the most likely correct configuration of the device during the configuration process. To this end, it may be trained in a multi-modal manner with text and images from many sources, in particular also from other medical networks, so as to provide a rich model which will likely predict the correct configuration.

The device may be configured as hardware and/or software. The hardware may include one or more processing units, storage, ports, buses, etc. The software may include one or more instructions to be executed on one or more processing units. The “method for configuring the device” is a computer-implemented method.

A “medical network” is an information technology (IT) network comprising two or more network components, such as computers, e.g., personal computers, laptops, servers, PLCs (programmable logic controller), etc. communicatively connected through a network connection (network technology) such as Ethernet, for example.

A “neural network” herein refers to an artificial neural network which is built up like a biological neural net, e.g., a human brain. In particular, an artificial neural network comprises an input layer and an output layer. It may further comprise a plurality of layers between the input and output layer. Each layer comprises at least one, preferably a plurality of nodes. Each node may be understood as a biological processing unit, e.g., a neuron. In other words, each neuron corresponds to an operation applied to input data. Nodes of one layer may be interconnected by edges or connections to nodes of other layers, in particular, by directed edges or connections. These edges or connections define the data flow between the nodes of the network. In particular, the edges or connections are equipped with a parameter, wherein the parameter is often denoted as “weight”. This parameter can regulate the importance of the output of a first node to the input of a second node, wherein the first node and the second node are connected by an edge.

Neural networks can be trained. “Self-supervised” learning (SSL) is a paradigm in machine learning where a model is trained on a task using the data itself to generate supervisory signals, rather than relying on external labels provided by humans. In the context of neural networks, self-supervised learning aims to leverage inherent structures or relationships within the input data to create meaningful training signals. SSL tasks are designed so that solving it requires capturing essential features or relationships in the data. The input data is typically augmented or transformed in a way that creates pairs of related samples. One sample serves as the input, and the other is used to formulate the supervisory signal. This augmentation can involve introducing noise, cropping, rotation, or other transformations. “Supervised” learning of a neural network is based on known pairs of input and output values, wherein the known input values are used as inputs of the neural network, and wherein the corresponding output value of the neural network is compared to the corresponding known output value. The artificial neural network independently learns and adapts, for example, using backpropagation, the weights for the individual nodes until the output values of the last network layer sufficiently correspond to the known output values according to the training data. For convolutional neural networks, this technique is also called “deep learning”.

“Configuration data” herein is to mean any data concerning the initial settings of the device, the network connection and/or at least one other device to make the device operable within the medical network. The configuration data may relate to ports of the device or the other device, protocols, standards or any other setup or initialization required in regard to the device, the network connection and/or at least one other device. The configuration data may relate to the device only, the network connection only or to the at least one other device only. Alternatively, the configuration data may relate to the device, the network connection and the at least one other device, for example. In some cases, adding the new device to the medical network may require configuration of the device, the network connection and/or on the at least one other device, as the case may be.

The neural network is pretrained prior to step a) using configuration data from a plurality of medical networks. The medical network or the medical networks may belong or be part of one or more health care providers such as clinics or hospitals, preferably. Pretraining comprises, preferably, training, in a self-supervised manner, the neural network using the configuration data from the plurality of medical networks. The neural network thus learns to understand typical configurations in medical networks before step a). Medical networks are known to comprise similar or largely similar devices, systems, and entities. Once the neural network has learnt from a sufficient number of medical networks, it will be able to make a good prediction at the correct configuration to be used in step c). This is particularly true as the (rich) neural network will be, prior to predicting the configuration in step c), be trained in step b) on the configuration data which is specific for the medical network at hand in step a). Thus, the neural network will gain an understanding of the medical network at hand and combine this knowledge with the knowledge accumulated in regard to the plurality of medical networks it has already been trained on in the past.

“Collecting” according to step a) may include harvesting or a mere identification of configuration data. It does not necessarily require storing the configuration data in a common storage.

During step c), the neural network (further) trained in step b) is used to predict a useful configuration of the device. As mentioned above, this configuration may relate to the setting of parameters or other (initial) settings required to make the device operable within the medical network and to thus be able to exchange medical data such as medical images, medical text data, such as reports, etc. with the at least one other device over the network connection.

According to an embodiment, step a) comprises collecting configuration data from one or more of the following sources: a user manual of the device, the network and/or the at least one other device, topology documentation of the network, in particular when the network is divided in two or more subnetworks, one or more communication standards applying to the device, the network and/or the at least one other device, e.g., DICOM, FHIR or HL7, and/or respective settings, e.g., stored in a configuration file, or protocols applying to the one or more communication standards, firewall settings, software licenses, operating system information or changes, cloud settings, authentication settings, one or more parameters set on a scanner, printer, workstation, electronic health record node or archive in the medical network.

The neural network is thus ideally trained on a vast amount of data which may also include public sources and other medical networks as explained above so as to best understand the domain of where it will be used. In this manner, the neural network will understand, inter alia, the configuration of software systems, TCP/IP networks, port mappings, compression algorithms, virtual private networks (VPN), network security, error messages and other real-world domain aspects.

Digital Imaging and Communication in Medicine (“DICOM”) is the well-established standard for defining the structure of diagnostic images such as Magnetic Resonance (MR), Computer-aided Tomography (CT) and Ultrasound. In addition to the image format, the standard also defines how to negotiate, query for, compress and transfer images.

Fast Healthcare Interoperability Resources (“FHIR”) is a standard for health care data exchange of clinical, diagnostic, medications, workflow, financial information in connection with health care.

Healthcare Level 7 (“HL7”) has developed the previous standards HL7 version 2 and 3, which standardizes the same message content as FHIR but with different architectures and technologies.

According to an embodiment, step a) comprises collecting configuration data from an HMI (Human Machine Interface) configuration file containing data regarding to a specific operator of the device, the network connection and/or the at least one other device.

Thereby, the neural network will learn HMI (Human Machine Interface) configuration data relating to a specific person, and thus will be able to reproduce the corresponding configuration on the device in step c).

According to an embodiment, the medical network is communicatively connected to a vendor network, the vendor network being configured to provide online services regarding the device, the network connection and/or the at least one other device, the online services including at least one of the following: software updates, artificial intelligence (AI) services, cloud data from other medical networks communicatively connected to the vendor network, and step a) comprises collecting configuration data from the vendor network.

Advantageously, data is not only collected from the medical network at hand, but also from other networks connected to the medical network such as a vendor network. This configuration data from the “external” network will be used in step b) to enrich the neural network even further.

According to a further embodiment, step a) comprises analyzing data traffic on the network connection and deriving the configuration data therefrom.

By learning from live data traffic in step b), even more suitable configuration data can be learnt by the neural network.

According to a further embodiment, the device, the network connection and/or the at least one other device are updated with a software update prior to step a).

This step ensures that all required updates are done before starting step a), thereby ensuring that the neural network learns from the latest configuration of the medical network.

According to a further embodiment, the device is a MR, CT, X-ray or ultrasound scanner, the network connection is an ethernet connection, and/or the at least one further device is a scanner, printer, workstation, electronic health record node, archive, router, or firewall.

A MR (Magnetic Resonance), CT (Computer Tomography), X-ray or ultrasound scanner usually comprises a scanning unit communicatively coupled with a processing unit for generating images based on signals generated from the scanning unit during scanning.

According to a further embodiment, the configured device is operated following step c) to perform a medical task.

The method for configuring a device within a medical network thus not only relates to the configuring, but also to putting that device to use in the medical network to the intended task. For example, a medical task can be performing an MR or CT scan.

According to a further embodiment, step c) comprises applying the trained neural network to a configuration interface of the device, network connection and/or at least one further device. For example, the configuration interface is a web interface. For example, the web interface may be based on the hypertext transfer protocol (HTTP).

According to a further embodiment, the configuration interface includes a least text, an image, and/or an HMI. Thereby, the neural network can learn the structure of the interface and make suitable suggestions for data fields to be filled out by a user.

According to a further embodiment, step c) comprises: guiding a user through an HMI when installing or updating the device in the medical network, providing a prompt through an HMI to a user for a query-answer interaction when installing or updating the device in the medical network, providing explanations through an HMI to a user when using a tooltip, prefilling fields in an HMI when installing or updating the device in the medical network and requesting a user to confirm the prefilled data before applying the prefilled data to the device, the network connection or the at least one other device, automatically configuring the device, the network connection and/or the at least one other device, and/or sending an authorization request to an authorization device for authorizing configuring the device, the network connection or the at least one other device.

According to a further embodiment, the trained unit is a foundational model and/or a multi-modal model.

A foundational neural network model is commonly a neural network model that is pretrained on a large amount of data, through which the model gains a broad understanding of its input domain. The training data (configuration data) may include one or more modalities, i.e. it may include image, text, audio, or video data, also in combination. Thus, the trained neural network may use, for example, an image of the current design of the medical network to therefrom derive configuration (for example, text) data relating to the device and which is used in step c) for the configuration of the device.

According to a further aspect, the invention relates to a computer program product comprising instructions which, when the program is executed by a computer, cause the computer to carry out the above-described method.

A computer program product, such as a computer program means, may be embodied as a memory card, USB stick, CD-ROM, DVD or as a file which may be downloaded from a server in a network. For example, such a file may be provided by transferring the file comprising the computer program product from a wireless communication network.

According to a further aspect, there is provided a system for configuring a device within a medical network, the medical network comprising at least one other device for communicating with the device via a network connection, the method comprising: a first unit for collecting configuration data regarding the device, the network connection and/or the at least one other device; a second unit for training, in a self-supervised manner, a neural network using the collected configuration data, the neural network being pretrained using configuration data from a plurality of medical networks; and a third unit for configuring the device depending on the trained neural network.

Herein, respective “units”, for example the first or second unit, may be implemented in hardware and/or software.

According to a further aspect, there is provided a device configured to be operable in a medical network, the medical network comprising at least one other device for communicating with the device via a network connection, the method comprising: a storage unit comprising configuration data obtained through applying the method as described above, and a processing unit for controlling the device depending on the configuration data.

The device is preferably a medical device such as a MR, CT, X-ray or ultrasound scanner.

Further possible implementations or alternative solutions of the invention also encompass combinations—that are not explicitly mentioned herein—of features described above or below with regard to the embodiments. The person skilled in the art may also add individual or isolated aspects and features to the most basic form of the invention.

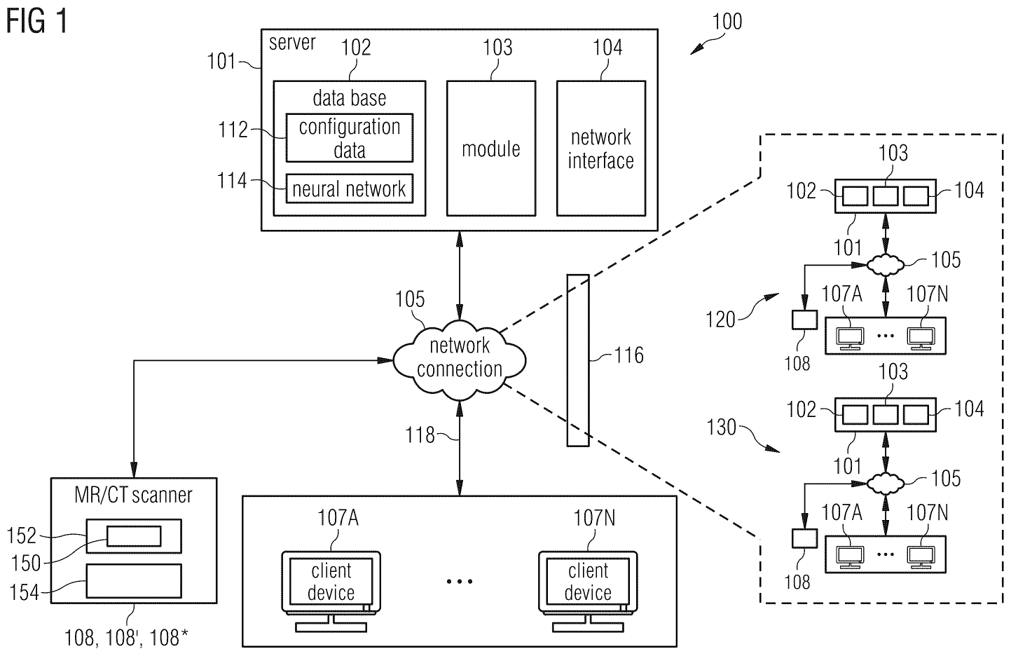

FIG. 1 shows a block diagram of a client-server architecture 100 embodying a medical network. The client-server architecture 100 comprises a server 101 and a plurality of client devices 107A-N. Each of the client devices 107A-N is connected to the server 101 via a network connection 105 (network technology such as ethernet) providing, for example, a local area network (LAN), a wide area network (WAN), Wi-Fi, etc. For example, the network connection 105 may connect the medical network 100 to a further medical network 120 as well as to a vendor network 130.

In one embodiment, the server 101 is deployed in a cloud computing environment. As used herein, “cloud computing environment” refers to a processing environment comprising configurable computing physical and logical resources, for example, networks, servers, storage, applications, services, etc., and data distributed over, for example, the internet. The cloud computing environment provides on-demand network access to a shared pool of the configurable computing physical and logical resources. The server 101 may include a database 102. The server 101 may further include a module 103 that is adapted to execute method steps to configure a device 108 within the medical network 100.

The client devices 107A-N are user devices, used by users, for example, medical personnel such as a radiologist, pathologist, physician, etc. In an embodiment, the user device 107A-N may be used by the user to receive medical images associated with the patient. The data can be accessed by the user via a graphical user interface of an end user web application on the user device 107A-N. In another embodiment, a request may be sent to the server 101 to access the medical images associated with the patient via the network connection 105.

An imaging unit 108 (one example of the present “device” or “medical device”) may be connected to the server 101 through the network connection 105. The imaging unit 108 may be a medical imaging unit 108 capable of acquiring a plurality of medical images. The medical imaging unit 108 may be, for example, a scanner unit (also termed “scanner” herein) such as a magnetic resonance imaging unit, computed tomography imaging unit, an X-ray fluoroscopy imaging unit, an ultrasound imaging unit, etc.

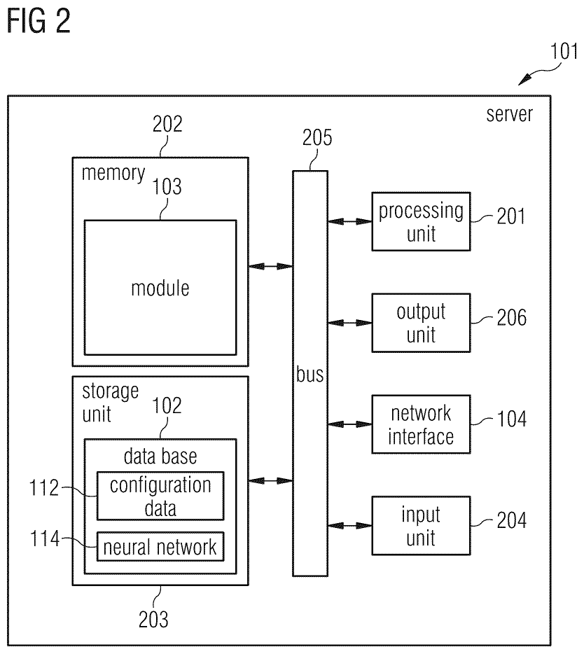

FIG. 2 is a block diagram of a data processing system 101 which may be implemented to execute method steps to configure a device 108 within the medical network 100. It is appreciated that the server 101 is an exemplary implementation of the system in FIG. 2. In FIG. 2, said data processing system 101 comprises a processing unit (or processor) 201, a memory 202, a storage unit 203, an input unit 204, an output unit 206, a bus 205, and a network interface 104.

The processing unit (or processor) 201, as used herein, means any type of computational circuit, such as, but not limited to, a microprocessor, microcontroller, complex instruction set computing microprocessor, reduced instruction set computing microprocessor, very long instruction word microprocessor, explicitly parallel instruction computing microprocessor, graphics processor, digital signal processor, or any other type of processing circuit. The processing unit 201 may also include embedded controllers, such as generic or programmable logic devices or arrays, application specific integrated circuits, single-chip computers, and the like.

The memory 202 may be a non-transitory memory device. The memory 202 may be coupled for communication with said processing unit 201. The processing unit 201 may execute instructions and/or code stored in the memory 202. A variety of computer-readable storage media may be stored in and accessed from said memory 202. The memory 202 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 202 comprises a module 103 stored in the form of machine-readable instructions on any of said above-mentioned storage media and may be in communication to and executed by processing unit 201. When executed by the processing unit 201, the module 103 causes the processing unit 201 to execute method steps to configure a device 108 within the medical network 100 as elaborated upon in detail in the following figures. The processing unit 201 and the memory 202 with the module 103 could also be implemented on any other device or entity within the medical network 100, e.g., on the device 108 (also on devices 108′, 108* as mentioned below), on any of the client devices 107A-N or in a cloud accessible through the network connection 105.

The storage unit 203 may be a non-transitory storage medium or memory device which stores the database 102. The input unit 204 may include input means such as keypad, touch-sensitive display, camera (such as a camera receiving gesture-based inputs), a port etc. capable of providing input signal such as a mouse input signal or a camera input signal. The bus 205 acts as interconnect between the processing unit 201, the memory 202, the storage unit 203, the input unit 204, the output unit 206 (e.g., a monitor or screen) and the network interface 104 (e.g., an ethernet port). The configuration data may be read into the database 102 via the network interface 104 or the input unit 204, for example.

Those of ordinary skill in the art will appreciate that said hardware depicted in FIG. 1 may vary for particular implementations. For example, other peripheral devices such as an optical disk drive and the like, Local Area Network (LAN)/ Wide Area Network (WAN)/Wireless (e.g., Wi-Fi) adapter, graphics adapter, disk controller, input/output (I/O) adapter also may be used in addition or in place of the hardware depicted. Said depicted example is provided for the purpose of explanation only and is not meant to imply architectural limitations with respect to the present disclosure.

A data processing system 101 in accordance with an embodiment of the present disclosure may comprise an operating system employing a graphical user interface (GUI). Said operating system permits multiple display windows to be presented in the graphical user interface simultaneously with each display window providing an interface to a different application or to a different instance of the same application. A cursor in said graphical user interface may be manipulated by a user through a pointing device. The position of the cursor may be changed and/or an event such as clicking a mouse button, generated to actuate a desired response.

One of various commercial operating systems, such as a version of Microsoft Windows™, a product of Microsoft Corporation located in Redmond, Washington may be employed if suitably modified. Said operating system is modified or created in accordance with the present disclosure as described.

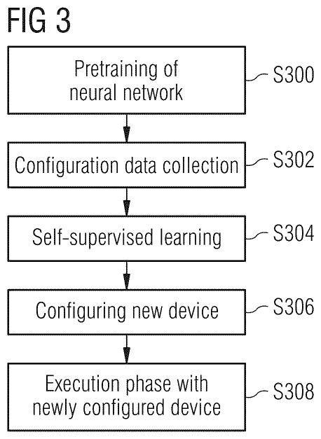

FIG. 3 shows a method for configuring a device within the medical network 100. For example, when the scanner 108 is replaced by a new model 108′ or when the scanner 108 is updated with a new software (the so updated device is indicated by reference numeral 108*), the scanner 108′, 108* (in particular a piece of software running on that scanner 108′) needs to be configured. However, configuring the device 108′, 108* may entail configuring the network connection 105 or one of the client devices 107A-N or the server 101 (also termed “at least one other device” herein). To simplify or fully automate this configuration process, method steps S300-S308 are provided.

In a first step S300, a neural network, in particular a large language model or a foundation model including a large language model, is trained using configuration data of medical networks such as the medical network 120, the vendor network 130, etc. This training is done using self-supervised learning, preferably. As this training is, in principle, the same as the training that will still be elaborated with regard to the medical network 100, training of the neural network on these other networks will not be elaborated any further for reasons of efficiency.

This pretrained neural network is stored as the neural network 114 (see FIGS. 1 and 2) in the database 102 stored on the server 101 (or any other computing device, such as a personal computer, a laptop, a PLC, etc.).

In a step S302, configuration data is collected all over the network 100 as well as from public sources available via the network connection 105 (such as the worldwide web or the like). For example, techniques such as data harvesting, web crawling, or the like may be used to collect the configuration data which is then stored as configuration data 112 in the database 102.

In many clinical settings, there is a divided network to consider, one part being on premise, the other part being outside in a cloud. Both parts are to be considered for collecting the configuration data 112 used during the training of the neural network 114 which may also provide context for a retrieval augmented generative model. Retrieval-augmented generation (RAG) is a technique for enhancing the accuracy and reliability of generative AI models with facts fetched from external sources.

For example, the medical network 120 may belong to the same hospital as the medical network 100 (protected by the same firewall in respect of the outside world). However, the medical network 100 is on premise, whereas the medical network 120 is arranged, at least partially, in a cloud. The networks 100, 120 are connected via the network connection 105.

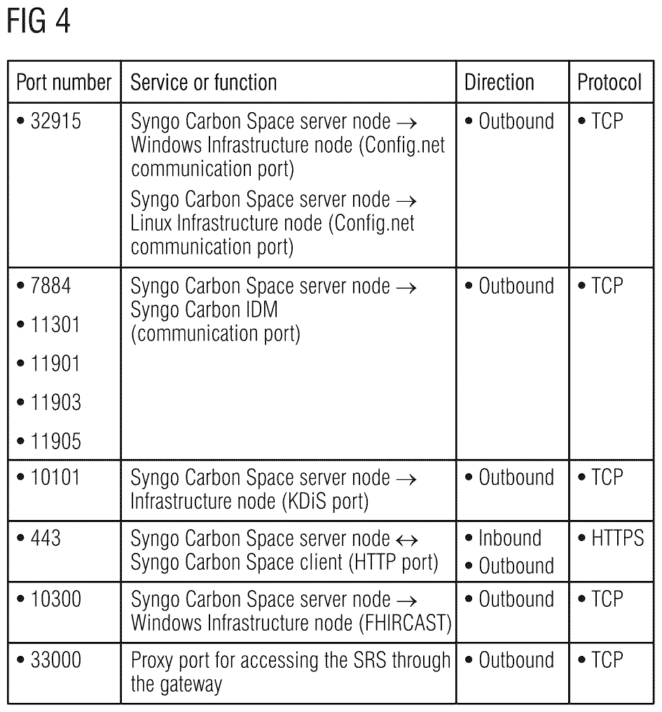

In some cases, it is preferable to update one or more entities within the medical network 100 before starting the data collection step S302. In connection with FIG. 4-11, examples of devices, systems and other entities are given which may be queried during the data collection step S302 for configuration data. This configuration data may be stored as configuration data 112 in the database 102 and then be used for self-supervised training of the neural network 114 in step S304 to, preferably, obtain a large model (foundation model), in particular a large language model which understands and generates language, images, video and/or audio (e.g., a transformer architecture may be used). “Collecting” may include the case where the configuration data is merely identified but not stored in one place.

According to an embodiment, data traffic 118 running over the network connection 105 is analyzed to understand which type of data, for example diagnostic image types, is sent between nodes in the medical network 100. Then, a mapping is done between the type of data and the type of nodes. Thereafter, all network nodes, systems or entities reached by the device 108 are queried for their capabilities. The result of this analysis and query may be saved as configuration data 112 and be used as training input for the neural network in step S304. By the same token, network logs may be used for network analysis (instead of the data traffic 118 itself). Network logs may be found, for example, on the firewall 116 (see FIG. 1) or any other network devices, such as switches (not shown).

Possible sources of configuration data will be elaborated in connection with FIGS. 4-11. For example, all available documentation and specification related to the new device 108′ or newly updated software (corresponding to device 108*) may be used. Further, if there are example configurations, descriptions of configuration fields or an API (Application Programming Interface) for entering configuration parameter information, then this configuration data may also be used to train the neural network 114. Additionally, the configuration data 112 could be given as context in the form of retrieval augmented generation without the need for additional training.

All information related to the medical network 100 such as the network plan and topology of the network may be collected as configuration data 112. This may in particular include the devices 107A-N, the scanner 108, the server 101, etc. arranged in the network 100 or the network connection 105 or network technology used. Further, this may include TCP/IP (Transmission Control Protocol/Internet Protocol) addresses of these devices, services which they support, port maps, the location of proxy and security firewalls and their configuration.

FIG. 4 shows an example of an excerpt of a port mapping in a handbook for an archive system. For example, a corresponding PDF (portable document format) may be stored on a local drive (storage device) of the client device 107A. This document will then be added to the configuration data 112 in the data collection step S302.

From FIG. 4, the (pretrained) neural network 114 learns, in a self-supervised manner, how the port number is related to the service or function, the direction of communication and the protocol used (see column headings of FIG. 4).

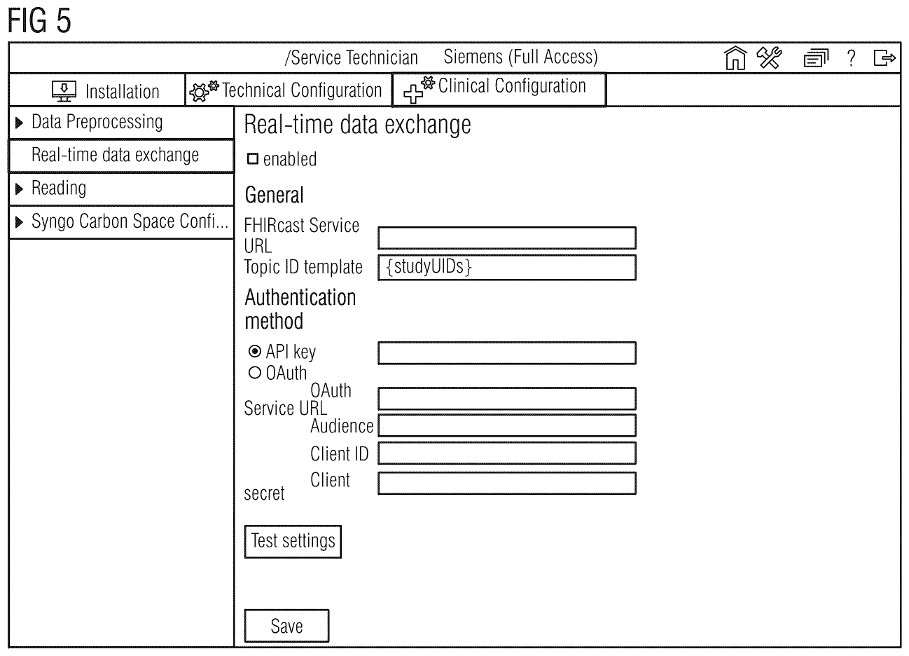

Another example of configuration data is shown in FIG. 5. FIG. 5 shows the configuration for connecting a DICOM (speaking) system and a fire/HL7 (speaking) system.

From FIG. 5, the neural network 114 learns the configuration for a real-time data exchange. Also, an authentication method may be learnt from FIG. 5. As in FIG. 4, the relevant information is text (modality) which can be integrated into a large language model.

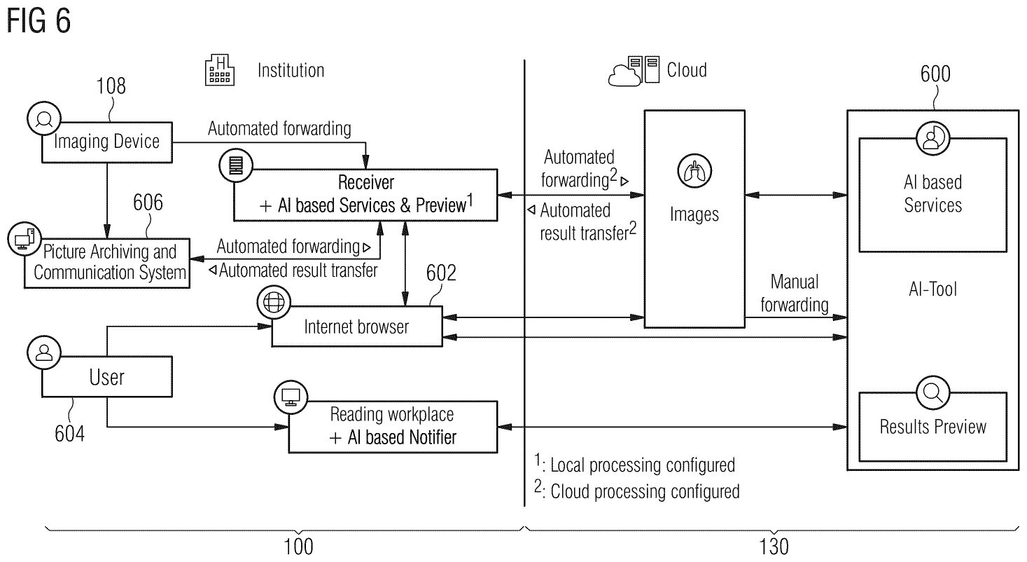

Now referring to FIG. 6, there is shown the medical network 100 being communicatively connected to the vendor network 130. As is typical in many clinical systems, there is a service contract between a software and/or hardware vendor and the clinic. The vendor network 130 provides a dedicated VPN tunnel into the medical network 100 (clinical network) to perform services on devices of the medical network 100. These services may include, for example, software updates or AI (Artificial Intelligence) services, etc. If contract legal requirements allow, configuration data 112 may also be collected within the vendor network 130 and save it in the database 102. Alternatively, the neural network 114 can directly learn from configuration data within the vendor network 130. For example, the vendor network 130 may include a trained neural network 600 which can be accessed by an Internet browser 602 by a user 604. The neural network 600 allows for an automated AI-based evaluation of images 606 generated by the scanner 108 (see FIG. 1) to identify, for example through segmentation or classification, an abnormality within the image data 606. The abnormality can be, for example, a specific type of disease, for example lung cancer, lung nodules, etc. For example, by learning the configuration of the neural network 600 in step S304, a desirable configuration of the scanner 108′, 108* may be learnt.

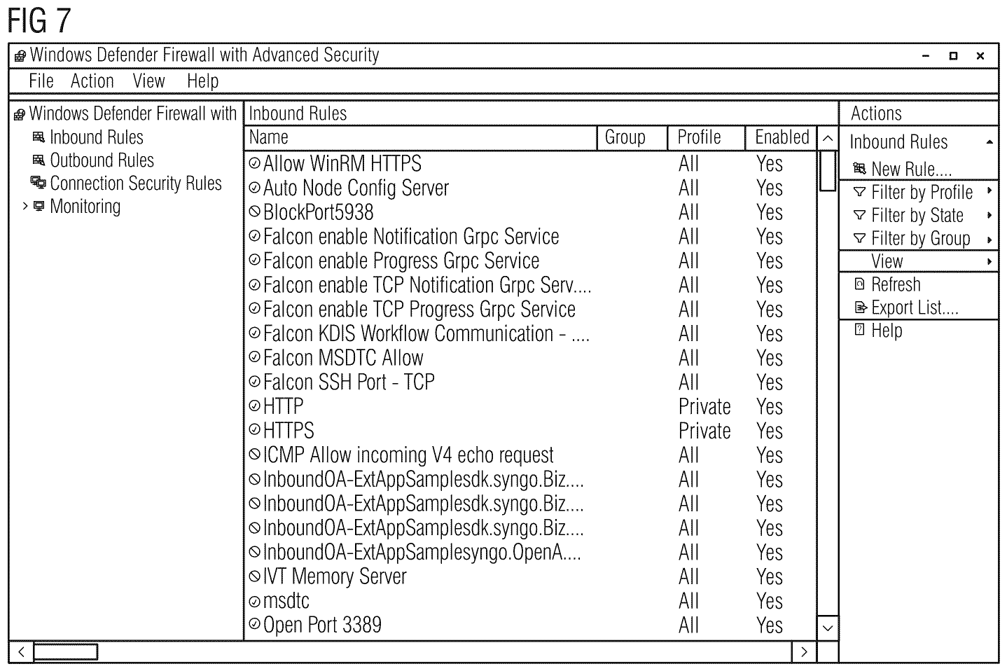

FIG. 7 shows further configuration data in the form of firewall rules. This configuration may, for example, be stored on the firewall 116 through which connection is made to an external medical network 120 or the vendor network 130.

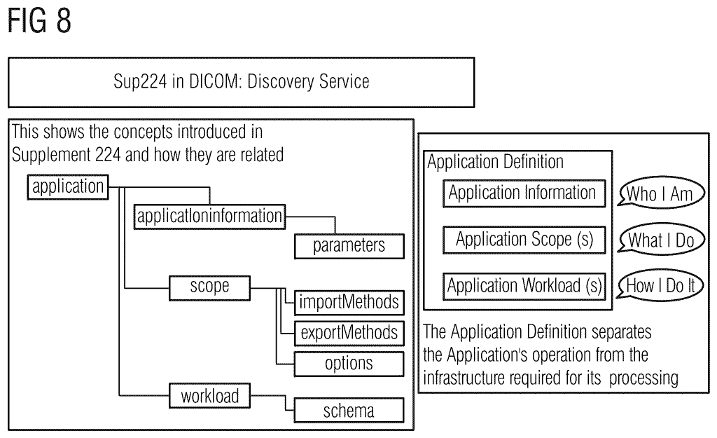

Configuration data 112 may be elicited from, for example, a DICOM discovery protocol as shown in FIG. 8, which interactively exposes the properties, services and capabilities of the system running the DICOM discovery protocol. The neural network 114 is trained in step S304 to understand this protocol and can be fed with many examples of variants of how this protocol is used in real-life scenarios.

For example, the below code line may be used to query a DICOM system:

### Example Query:

findscu -v -P -k 0008,0052=IMAGE -aet YOUR_AE_TITLE -aec THEIR_AE_TITLE -aec THEIR_AE_TITLE HOSTNAME_OR_IP PORT

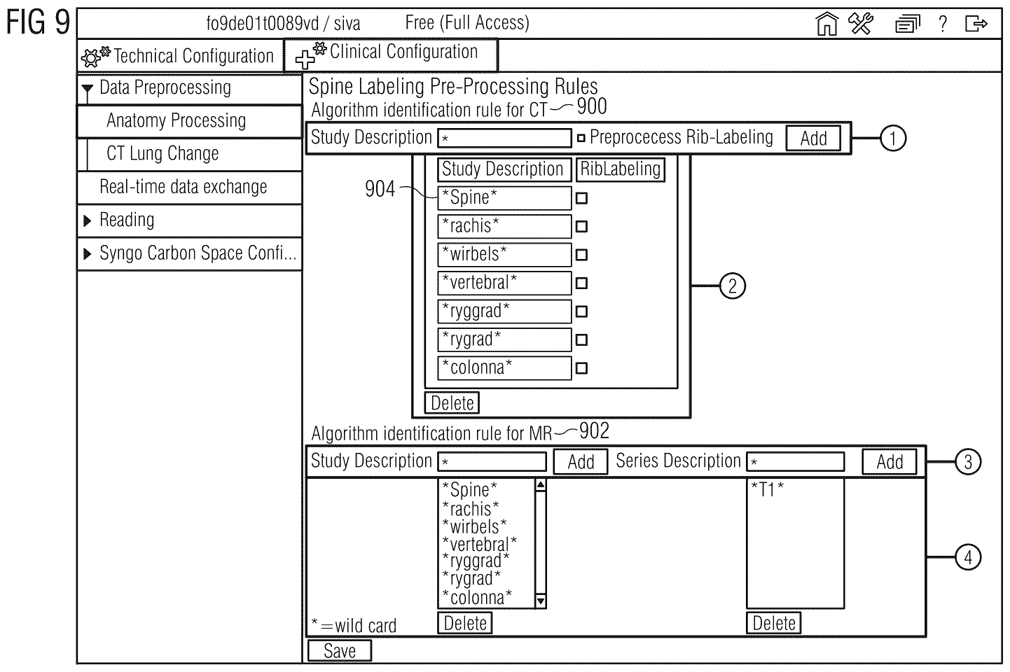

FIG. 9 illustrates a (e.g., web) user interface (one example of an HMI) comprising configuration data on preprocessing rules. For example, different preprocessing rules 900 and 902 may be implemented for CT on the one hand and MR on the other hand. For example, depending on the selected preprocessing rule, labels in regard to images each showing a spine are searched automatically for certain keywords 904, for example, “spine”, “rachis”, etc. Also, the setting of these preprocessing rules represents configuration data in the sense of the present application and can be used to train the neural network 114.

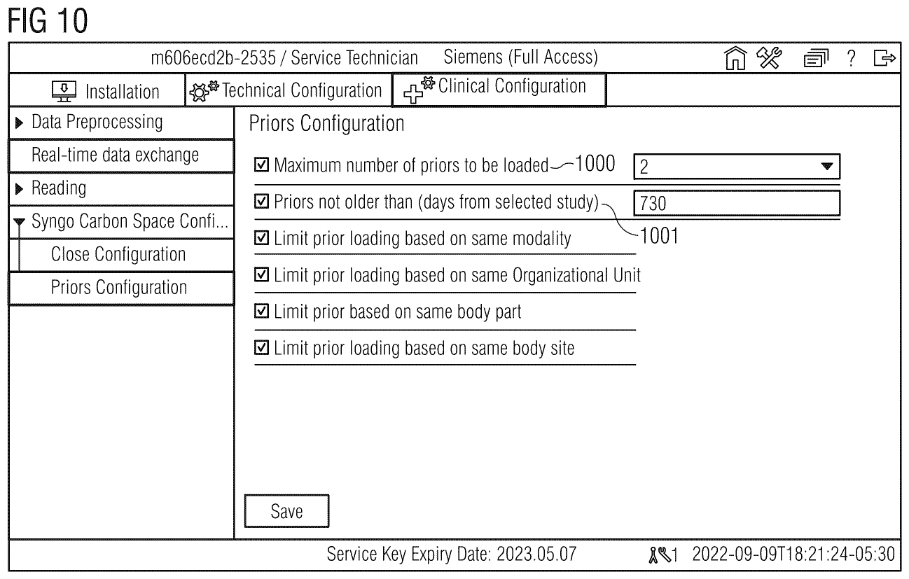

FIG. 10 shows an example of a configuration of a (e.g., web) user interface for handling of prior clinical studies. For example, different options 1000, 1001 can be selected to limit the number of prior images or select images from a certain date onwards as the standard configuration for priors. “Priors” are collections, called “studies”, of diagnostic images and attached reports containing findings and conclusions made from the radiologist reading the images at the time of the study. Each study is done at a specific date in the past. For example, to follow up on a specific cancer treatment development several prior imaging studies are evaluated to understand the development of the cancer and the success of the treatment. Again, this represents configuration data 112 in the sense of the present application.

The settings 1000, 1001 may be a user-specific (operator-specific) data set (representing configuration data 114) that will have different values for different operators. Thus, the neural network 114 can take into account, for the configuration of the new device 108′ or updated device 108* in step S306, user-specific preferences which saves end users time to customize HMIs in accordance with their personal preferences.

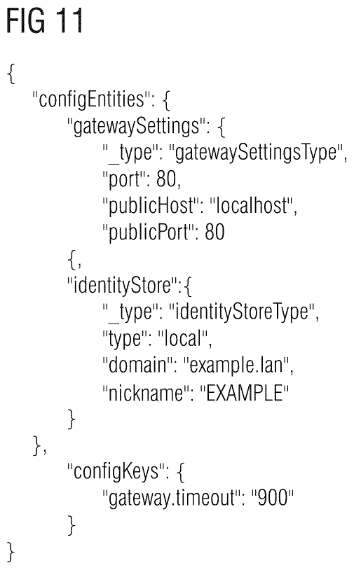

FIG. 11 illustrates a configuration text file of a gateway which is collected and used in the training of the neural network 114 in steps S302 and S304.

Further possible sources of configuration data collected in step S302 and used in step S304 are:
EHR (electronic health record) nodes
DICOM nodes: printers, archives, workstations
DICOM scanner nodes with scan parameters
Authentication information
Licenses
Additional software installations
Preprocessing rules
Layout configurations
Firewall settings
OS (operating system) related changes
Cloud accounts and registrations
Architecture optimizations

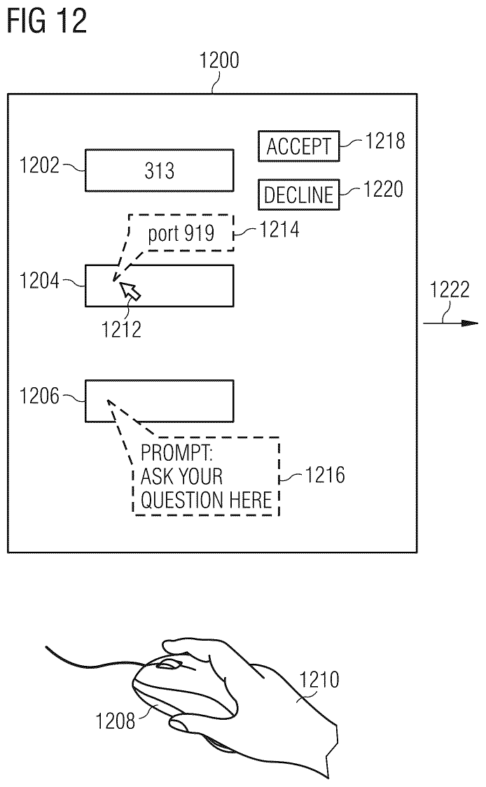

FIG. 12 shows different ways of implementing the configuration step S306 of FIG. 3. At this point, the trained neural network 114 is applied to configure the device 108′, 108*. This is done for example by applying the neural network 114 to a (e.g. web) user interface 1200 of the device 108′, 108* for configuring the same. Preferably, the user interface 1200 comprises information which identifies the device 108′, 108* to the neural network 114 such as e.g. a heading “CT scanner 1234 set-up wizard”, with “1234” being the identifier.

For example, FIG. 12 shows the user interface 1200 (a form) with fields 1202, 1204 and 1206. Each of these fields is to be filled with configuration data. Once this configuration data has been filled in and the web interface 1200 has been sent off, the corresponding parameters are set on the device 108′, 108*, within the network connection 105 or on any other device, such as the client devices 107A-107N or the server 101. For example, the user interface 1200 is controlled with a mouse 1208.

In the case of the field 1202, the neural network 114 automatically fills this field with a proposed parameter “313”. The user 1210 is then, through a corresponding software routine, requested to accept (1218) or decline (1220) the proposed parameter “313”.

The field 1204 is not filled automatically, but once the cursor 1212, controlled by the mouse 1208, hovers over the field 1204, a tooltip 1214 is displaced which shows a proposed parameter “919” to be entered into the field 1204 on mouse click.

The field 1206 is also not filled automatically, but a prompt is displayed in a dialogue box 1216. Here, the user 1210 can type a query to the neural network 114 which will then be answered by the neural network 114 and will help the user 1210 to correctly fill in the field 1206. Using the prompt, the user 1210 is guided through the configuration interactively. The result of the “conversation” is used as entries into the field 1206.

In yet another embodiment, the neural network 114 is applied to the configuration data 112 and then automatically sets parameters on one, some or all of the devices within the medical network 100, in particular on the device 108′, 108* newly added or updated.

In step S306 (FIG. 3), the trained neural network 114 is applied to the configuration data 112 so as to produce configuration data 150 (see FIG. 1) for configuring the new scanner 108′ or the existing scanner 108* including updated software. The configuration data 150 is stored on the storage device 152 of the scanner 108, 108′. The scanner 108′, 108* further has a processing unit 154 configured for controlling the scanner 108′, 108* depending on the configuration data 150 to perform a medical task in step S308, in particular a scanning task such as to produce an MR or CT image and to transfer that image via the network connection 105 to the server 101 or to one of the client devices 107A-N.

It may be provided as shown in FIG. 12 that once any of the fields 1202, 1204 or 1206 has been filled and the form 1200 has been sent off, the parameters on the device 108′, 108* are not set automatically (the configuration file 150 is not immediately generated), but an authorization request 1222 is sent to the server 101 for authorizing the configuration of the device 108′, 108*. In one example, an IT administrator has to authorize the requested configuration 150 of the scanner 108′, 108* before it is implemented.

Although the present invention has been described in accordance with preferred embodiments, it is obvious for the person skilled in the art that modifications are possible in all embodiments.

### LIST OF REFERENCE NUMERALS

100 client-server architecture
101 server
102 database
103 module
104 network interface
105 network connection
107A-N device
108, 108′, 108* device
112 configuration data
114 neural network
116 firewall
118 data traffic
120 further medical network
130 vendor network
150 configuration data
152 storage device
154 processing device
201 processing unit
202 memory
203 storage unit
204 input unit
205 bus
206 output unit
600 trained neural network
602 internet browser
604 user
606 images
900 preprocessing rule
902 preprocessing rule
904 keywords
1000 option
1001 option
1200 web interface
1202 field
1204 field
1206 field
1208 mouse
1210 user
1212 cursor
1214 tooltip
1216 dialogue
1218 accept
1220 decline
1222 authorization request
S300-S308 method steps

1. A method for configuring a device within a medical network, the medical network comprising at least one other device for communicating with the device via a network connection, the method comprising:
a) collecting configuration data regarding the device, the network connection, the at least one other device, or a combination thereof;
b) training, in a self-supervised manner, a neural network using the collected configuration data, the neural network being pretrained prior to step a) using configuration data from a plurality of medical networks; and
c) configuring the device depending on the trained neural network.

2. The method according to claim 1, wherein step a) comprises collecting the configuration data from
a user manual of the device, the medical network, the at least one other device, or a combination thereof,
topology documentation of the medical network,
one or more communication standards or settings applying to the device, the medical network, the at least one other device, or a combination thereof, or
protocols applying to the one or more communication standards, firewall settings, software licenses, operating system information or changes, cloud settings, authentication settings, one or more parameters set on a scanner, printer, workstation, electronic health record node or archive in the medical network, or a combination thereof.

3. The method according to claim 1, wherein step a) comprises collecting the configuration data from a Human Machine Interface (HMI) configuration file containing data relating to a specific operator of the device, the network connection, the at least one other device, or a combination thereof.

4. The method according to claim 1, wherein:
the medical network is communicatively connected to a vendor network, the vendor network being configured to provide online services regarding the device, the network connection, the at least one other device, or a combination thereof, and
step a) comprises collecting configuration data from the vendor network.

5. The method according to claim 4 wherein the online services comprise software updates, artificial intelligence (AI) services, cloud data from other medical networks communicatively connected to the vendor network, or a combination thereof.

6. The method according to claim 1, wherein step a) comprises analyzing data traffic on the network connection and deriving the configuration data therefrom.

7. The method according to claim 1, further comprising updating, with a software update prior to step a), the device, the network connection, the at least one other device, or a combination thereof.

8. The method according to claim 1, wherein the device comprises a magnetic resonance (MR), computed tomographic (CT), X-ray or ultrasound scanner.

9. The method according to claim 1, wherein the network connection comprises an ethernet connection.

10. The method according to claim 1, wherein the at least one other device comprises a scanner, printer, workstation, electronic health record node, archive, router, or firewall.

11. The method according to claim 1, wherein the configured device is operated following step c) to perform a medical task.

12. The method according to claim 1, wherein step c) comprises applying the trained neural network to a configuration interface of the device, a network connection, at least one further device, or a combination thereof.

13. The method according to claim 12, wherein the configuration interface is a web interface and includes text, an image, an HMI, or a combination thereof.

14. The method according to claim 1, wherein step c) comprises:
guiding a user through an HMI when installing or updating the device in the medical network,
providing a prompt through an HMI to the user for a query-answer interaction when installing or updating the device in the medical network,
providing explanations through an HMI to the user when using a tooltip,
prefilling fields in an HMI when installing or updating the device in the medical network and requesting the user to confirm the prefilled data before applying the prefilled data to the device, the network connection, the at least one other device, or a combination thereof,
automatically configuring the device, the network connection, the at least one other device, or a combination thereof, or
sending an authorization request to an authorization device for authorizing configuring the device, the network connection, the at least one other device, or a combination thereof.

15. The method according to claim 1, wherein the trained neural network is a foundation model, a multi-modal model, or a combination thereof.

16. A system for configuring a device within a medical network, the medical network comprising at least one other device for communicating with the device via a network connection, the system comprising:
a non-transitory memory device for storing computer readable program code; and
a processor in communication with the non-transitory memory device, the processor being operative with the computer readable program code to perform steps including
collecting configuration data regarding the device, the network connection, the at least one other device, or a combination thereof,
training, in a self-supervised manner, a neural network using the collected configuration data, wherein the neural network is pretrained using configuration data from a plurality of medical networks, and
configuring the device depending on the trained neural network.

17. The system according to claim 16, wherein the processor is operative with the computer readable program code to collect the configuration data regarding the device, the network connection, the at least one other device, or a combination thereof, by analyzing data traffic on the network connection and deriving the configuration data therefrom.

18. The system according to claim 16, wherein the processor is operative with the computer readable program code to configure the device depending on the trained neural network by applying the trained neural network to a configuration interface of the device, a network connection, at least one further device, or a combination thereof.

19. The system according to claim 16, wherein the trained neural network is a foundation model, a multi-modal model, or a combination thereof.

20. One or more non-transitory computer-readable media comprising computer-readable instructions, that when executed by a processor, cause the processor to perform steps comprising:
collecting configuration data regarding the device, the network connection, the at least one other device, or a combination thereof;
training, in a self-supervised manner, a neural network using the collected configuration data, wherein the neural network is pretrained using configuration data from a plurality of medical networks; and
configuring the device depending on the trained neural network.
